(19) United States
(12) Patent Application Publication
(10) Pub. No.: US 2021/0000708 A1
(43) Pub. Date: Jan. 7, 2021

(54) MAGNETIC EYE LINE, LIQUID EYELINER AND GEL EYELINER, AND MAGNETIC ADSORPTION-TYPE FALSE EYELASH CORRESPONDING THERETO AND WEARING METHOD THEREFOR

(71) Applicant: APR Beauty Group Inc., Toronto (CA)

(72) Inventors: Jun SHEN, New Westminster (CA); Min-Yan ZHENG, Richmond Hill (CA); Fengying FU, Markham (CA)

(21) Appl. No.: 16/503,750

(22) Filed: Jul. 5, 2019

(51) Int. Cl. A61K 8/19; A41G 5/02; A61K 8/25; A61K 8/34; A61K 8/58; A61K 8/49; A61K 8/44; A61K 8/92; A61Q 1/10

(52) U.S. Cl. CPC A61K 8/19; A41G 5/02; A61K 8/25; A61K 8/345; A61Q 1/10; A61K 8/585; A61K 8/4946; A61K 8/44; A61K 8/922; A61K 8/34

(57) ABSTRACT

Disclosed are a magnetic eye line, a liquid eyeliner and a gel eyeliner, and a false eyelash corresponding thereto and a wearing method therefor, wherein the eye line contains iron oxide black, and a plurality of small magnets are evenly distributed along a stem of the false eyelash. The liquid or gel eyeliner disclosed in the present invention is resistant to water, sweat and oils after application, is long lasting and smudge-proof, and has no side effects on the eye skin; magnetic eye lines drawn thereby contain iron oxide black as a magnetic substance, have a stronger magnetic property and can adsorb the false eyelash with small magnets, such that the process of wearing the false eyelash is simple, and the false eyelash is aesthetic and does not easily separate after wearing, and is environmentally friendly and safe.

### TECHNICAL FIELD

The present invention belongs to the field of cosmetics, and particularly relates to a magnetic eye line in the field, and a magnetic adsorption-type false eyelash corresponding thereto and a wearing method therefor.

### BACKGROUND ART

The wearing of traditional false eyelashes necessarily requires the use of a specialized false eyelash glue, which not only results in loose fixation and ease of fall off, but also indicates that the glue is irritating to the skin around the eyes, and is thus unsuitable for people with sensitive skin and sensitive eyes. In recent years, there have also been several revolutionary inventions, for example, Chinese invention patent application CN 207767606 U discloses a magnetic adsorption-type false eyelash, which is divided into an upper piece and a lower piece, wherein stems of the upper and lower pieces are respectively attached with different magnets with positive and negative poles, and the wearing thereof requires particularly skilled operation or the use of a specialized wearing tool of patent CN 108903106 A, causing wearing difficulties; in addition, since it is made up of two pieces, relatively great heaviness is caused after wearing, and a foreign body sensation is caused; furthermore, it is not aesthetic due to the presence of too many magnets, which is contrary to the original intention of wearing the false eyelash for aesthetics.

### SUMMARY OF THE INVENTION

The technical problem to be solved by the present invention is to provide a magnetic eye line, a liquid eyeliner and a gel eyeliner, which do not require any glue, and a magnetic adsorption-type false eyelash corresponding thereto and a wearing method therefor.

The present invention is implemented by means of the following technical solutions:

A magnetic eye line, an improvement for which lies in that the eye line contains iron oxide black.

Furthermore, the purity of iron oxide black is 99.99%.

A magnetic eye line, an improvement therefor lies in that it comprises the following components by weight: 35.6% of iron oxide black; 4% of 1,2-propanediol; 29.6% of decamethylcyclopentasiloxane; 29.6% of a reaction product of sodium silicate with chlorotrimethylsilane and isopropanol; 1% of tetrakis(trimethylsiloxy)silane; 0.1% of bis(hydroxymethyl)imidazolidinyl urea; and 0.1% of IPBC.

A magnetic gel eye line, an improvement there for lies in that it comprises the following components by weight: 30% of iron oxide black; 10% of olive oil; 15% of decamethylcyclopentasiloxane; 15% of a reaction product of sodium silicate with chlorotrimethylsilane and isopropanol; 0.5% of tetrakis(trimethylsiloxy)silane; 29.3% of beeswax, 0.1% of bis(hydroxymethyl)imidazolidinyl urea; and 0.1% of IPBC.

A magnetic adsorption-type false eyelash corresponding to the magnetic eye line as described above, an improvement therefor lies in that a plurality of small magnets are evenly distributed along a stem of the false eyelash.

Furthermore, the stem of said false eyelash is a soft stem, a hard stem or a cotton stem.

Furthermore, said small magnets are attached to the stem of the false eyelash.

Furthermore, said small magnets are orderly and regularly arranged in a row along the stem of the false eyelash.

A method for wearing a false eyelash, involving using the magnetic eye line and the magnetic adsorption-type false eyelash, as described above, improvements therefor lie in that it comprises the following steps:

(1) drawing a magnetic eye line using a magnetic liquid eyeliner or gel eyeliner; and

(2) aligning the small magnets at the stem of the magnetic adsorption-type false eyelash with the magnetic eye line drawn in step (1), so that the false eyelash can be adsorbed to the magnetic eye line based on the magnetic principle, thereby completing the wearing.

Furthermore, after the false eyelash is adsorbed to the magnetic eye line in step (2), the position of the false eyelash on the magnetic eye line can also be adjusted.

The beneficial effects of the present invention are as follows:

the magnetic liquid eyeliner disclosed in the present invention can be used for common eyeliner pencils, and the gel eyeliner may be used for bottled or boxed eye line tools, which eyeliners are resistant to water, sweat and oils after application, are long lasting and smudge-proof, and have no side effects on the eye skin; magnetic eye lines drawn thereby contain iron oxide black as a magnetic substance, have a stronger magnetic property and can adsorb the false eyelash with small magnets, such that the process of wearing the false eyelash is simple, and the false eyelash is aesthetic and does not easily separate after wearing, and is environmentally friendly and safe, this representing a milestone creation in the process of the development of false eyelash wearing.

The magnetic adsorption-type false eyelash disclosed in the present invention has the characteristics of firmness, no glue involved, being easy to handle and wear, etc., and the false eyelash having magnets attached at the stem can be repeatedly worn as long as it has enough magnetism; the false eyelash is not only simple, convenient and easy to wear, but is also environmentally friendly, mild and non-irritating. After being worn, the false eyelash gives lightness without any discomfort and foreign body sensation, the false eyelash is perfectly and tightly integrated with and adsorbed to the eye line, and it is neat and not easy to fall off, thereby resulting in complete replacement of false eyelash glues, solving the problem of many people with aesthetic appreciation having no chance to wear false eyelashes due to the fact that the eyes are sensitive to glues, and also avoiding the embarrassment of the glue easily failing. The magnetic adsorption-type false eyelash disclosed in the present invention also solves the problems of the two-piece magnetic adsorption-type false eyelash having difficulties in wearing, being heavy and having low aesthetic quality.

The method for wearing a false eyelash as disclosed in the present invention achieves both aims at once, i.e. realizing direct wearing of the false eyelash as long as an eye line is drawn, thereby saving makeup steps.

### BRIEF DESCRIPTION OF THE DRAWINGS

FIG. 1 is a schematic structural view of a magnetic adsorption-type false eyelash disclosed in example 1 of the present invention;

FIG. 2 is a schematic view of step (1) in the wearing method disclosed in example 1 of the present invention; and

FIG. 3 is a schematic view of step (2) in the wearing method disclosed in example 1 of the present invention.

### DETAILED DESCRIPTION OF EMBODIMENTS

In order to make the objectives, technical solutions and advantages of the present invention clearer, the present invention will be further described in detail below with reference to the accompanying drawings and examples. It is to be understood that the specific examples described herein are merely illustrative of the invention but are not intended to limit the invention.

Example 1: this example discloses a magnetic eye line containing iron oxide black having a purity of 99.99%. This example also discloses a magnetic liquid eyeliner comprising the following components by weight: 35.6% of iron oxide black; 4% of 1,2-propanediol; 29.6% of decamethylcyclopentasiloxane; 29.6% of a reaction product of sodium silicate with chlorotrimethylsilane and isopropanol; 1% of tetrakis(trimethylsiloxy)silane; 0.1% of bis(hydroxymethyl)imidazolidinyl urea; and 0.1% of IPBC. In the magnetic liquid eyeliner, the purity of iron oxide black is 99.99%.

This example also discloses a magnetic gel eyeliner comprising the following components by weight: 30% of iron oxide black; 10% of olive oil; 15% of decamethylcyclopentasiloxane; 15% of a reaction product of sodium silicate with chlorotrimethylsilane and isopropanol; 0.5% of tetrakis(trimethylsiloxy)silane; 29.3% of beeswax, 0.1% of bis(hydroxymethyl)imidazolidinyl urea; and 0.1% of IPBC. In the magnetic gel eyeliner, the purity of iron oxide black is 99.99%. The magnetic liquid eyeliner or magnetic gel eyeliner is a medium rich in iron oxide black as a magnetic substance, and can be used for drawing a magnetic eye line by means of an eyeliner pencil or a boxed gel eyeliner.

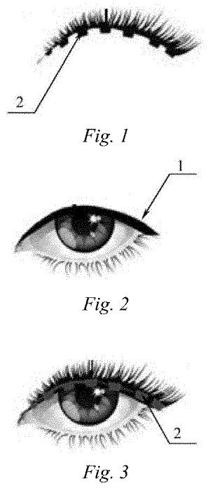

As shown in FIG. 1, this example also discloses a magnetic adsorption-type false eyelash corresponding to the magnetic eye line as described above, and a plurality of small magnets 2 are evenly distributed along the stem of the false eyelash. Said small magnets are attached to the stem of the false eyelash, and are orderly and regularly arranged in a row along the stem of the false eyelash.

In this example, said false eyelash may involve various stems including soft stems, hard stems or cotton stems, etc. of various lengths and various types.

This example also discloses a method for wearing a false eyelash, involving using the magnetic eye line and the magnetic adsorption-type false eyelash as described above, comprising the following steps:

(1) drawing a magnetic eye line 1 using a magnetic liquid eyeliner or gel eyeliner, as shown in FIG. 2; and

(2) aligning the small magnets 2 at the stem of the magnetic adsorption-type false eyelash with the magnetic eye line drawn in step (1), as shown in FIG. 3, so that the false eyelash can be adsorbed to the magnetic eye line based on magnetic principles, thereby completing the wearing.

Specifically, a spontaneous magnetization zone is formed between the false eyelash, to which the small magnets are attached, and the magnetic eye line, and the ferromagnetic substance (iron oxide black) in the liquid eyeliner or the gel eyeliner, once being magnetized by means of magnetic principles, produces a stronger attraction force with the small magnets, whereby the magnetic eye line is firmly bonded to the false eyelash attached with the small magnets. After the false eyelash is adsorbed to the magnetic eye line in step (2), the position of the false eyelash on the magnetic eye line may also be adjusted according to requirements for comfortableness and aesthetic quality.

1. A magnetic eye line, characterized in that the eye line contains iron oxide black.

2. The magnetic eye line according to claim 1, characterized in that the purity of iron oxide black is 99.99%.

3. A magnetic liquid eyeliner, characterized by comprising the following components by weight: 35.6% of iron oxide black; 4% of 1,2-propanediol; 29.6% of decamethylcyclopentasiloxane; 29.6% of a reaction product of sodium silicate with chlorotrimethylsilane and isopropanol; 1% of tetrakis(trimethylsiloxy)silane; 0.1% of bis(hydroxymethyl)imidazolidinyl urea; and 0.1% of IPBC.

4. A magnetic gel eyeliner, characterized by comprising the following components by weight: 30% of iron oxide black; 10% of olive oil; 15% of decamethylcyclopentasiloxane; 15% of a reaction product of sodium silicate with chlorotrimethylsilane and isopropanol; 0.5% of tetrakis(trimethylsiloxy)silane; 29.3% of beeswax, 0.1% of bis(hydroxymethyl)imidazolidinyl urea; and 0.1% of IPBC.

5. A magnetic adsorption-type false eyelash corresponding to the magnetic eye line of claim 1, characterized in that a plurality of small magnets are evenly distributed along a stem of the false eyelash.

6. The magnetic adsorption-type false eyelash according to claim 5, characterized in that the stem of said false eyelash is a soft stem, a hard stem or a cotton stem.

7. The magnetic adsorption-type false eyelash according to claim 5, characterized in that said small magnets are attached to the stem of the false eyelash.

8. The magnetic adsorption-type false eyelash according to claim 5, characterized in that said small magnets are orderly and regularly arranged in a row along the stem of the false eyelash.

9. A method for wearing a false eyelash, involving using the magnetic eye line of claim 1 and the magnetic adsorption-type false eyelash of claim 5, characterized by comprising the following steps:
(1) drawing a magnetic eye line using a magnetic liquid eyeliner or gel eyeliner; and
(2) aligning the small magnets at the stem of the magnetic adsorption-type false eyelash with the magnetic eye line drawn in step (1), so that the false eyelash can be adsorbed to the magnetic eye line based on magnetic principles, thereby completing the wearing.

10. The method for wearing a false eyelash according to claim 9, characterized in that after the false eyelash is adsorbed to the magnetic eye line in step (2), the position of the false eyelash on the magnetic eye line can also be adjusted.
